SEQUENCE LISTING (3 sequences)

SEQ ID NO: 1 - DNA, 3885 bp, Artificial
  Dox-regulable expression casette
gcggccgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattcaccatgtcaagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatccgtcgacaagcttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggtctagtcgaggccctttcgtcttcacacgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccagcatggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagcggccgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagagggccctattctattagtggtcacctaaatggcgacgcgtgctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcctctagcttcctcgctctag

SEQ ID NO: 2 - RNA, 16 nt, Artificial
  siRNA2
aggaccuaacaaaguu

SEQ ID NO: 3 - RNA, 19 nt, Artificial
  siRNA4
guacagggauaaacauuac

(19) United States
(12) Patent Application Publication
(10) Pub. No.: US 2010/0311809 A1
(43) Pub. Date: Dec. 9, 2010

(54) VIRAL VECTOR SYSTEM, A COMPOSITION COMPRISING THE VIRAL VECTOR SYSTEM AND ITS USE

(75) Inventors: Henry Fechner, Luckau (DE); Sandra Pinkert, Leipzig (DE); Jens Kurreck, Berlin (DE); Denise Werk, Berlin (DE)

Correspondence Address: Workman Nydegger;1000 Eagle Gate Tower, 60 East South Temple, Salt Lake City, UT, 84111, US

(73) Assignee: CHARITE UNIVERSITAETSMEDIZIN BERLIN, Berlin (DE)

(21) Appl. No.: 12/680,425

(22) PCT Filed: Oct. 27, 2008
(86) PCT No.: PCT/EP2008/009058

§ 371 Date: Jun. 24, 2010

(30) Foreign Application Priority Data
Oct. 26, 2007 (DE) 10 2007 052 005.2

(51) Int. Cl. A61K 31/713; C12N 15/63; A61P 31/14

(52) U.S. Cl. 514/44 A; 435/320.1

(57) ABSTRACT

The present invention relates to a viral vector system comprising at least one viral vector and at least one regulable expression cassette inserted in said viral vector applicable for the treatment of virally infected cells. Preferably, the at least one regulable expression cassette comprises at least one transactivator, at least one promoter and at least one nucleotide sequence coding for a transgene, preferably a fusion protein. The present invention also relates to a composition comprising said viral vector and antiviral siRNAs for treatment of virally infected cells.

### CROSS-REFERENCE TO A RELATED APPLICATION

This application is a National Phase patent application of International Patent Application Number PCT/EP2008/009058, filed on Oct. 27, 2008, which claims priority of German Patent Application Number 10 2007 052 005.2, filed on Oct. 26, 2007.

### BACKGROUND

The invention relates to a vector system a and a composition for treatment of virally infected cells.

Enteroviruses such as coxsackievirus, poliovirus and echovirus are small non-enveloped viruses belonging to the picornavirus family. They possess a single-stranded RNA genome in positive orientation that acts directly as mRNA in infected cells.

Picornaviruses are separated into nine distinct genera and include many important pathogens of humans and animals. The diseases they cause are varied, ranging from acute “common-cold”-like illnesses, to poliomyelitis, hepatitis to chronic infections in livestock like food-and-mouth disease. Two main categories are enteroviruses and rhinoviruses.

Thus, Picornaviruses are of high clinical relevance. However, currently there is no specific therapy available.

Coxsackievirus B3 (CVB3), a member of the enterovirus group of the Picornaviridae, is one of the most commonly identified infectious agents associated with acute and chronic myocarditis, and can also mediate infectious pancreatitis and meningitis. The virus is especially critical for newborn and babies.

Acute enterovirus myocarditis may not lead to initial mortality. In some cases, acute myocarditis can persist chronically and develop into a dilated cardiomyopathy (DCM), which is one of the most frequent causes of heart transplantation. In biopsies of DCM patients both persistent and latent enterovirus infections were detected. Cultured human foetal heart cells infected with CVB-3 showed completely lysed myocytes within a few days, whereas myocardial fibroblasts survived and multiplied. Continuous production of CVB-3 indicated a carrier state infection of human myocardial fibroblasts.

Currently, enterovirus myocarditis is treated non-specifically by conventional supportive methods, since no effective antiviral therapy is available. CVB load, replication and persistence are directly associated with cardiac injury and progression of the disease. Direct cytopathogenic effect of CVB in vitro, and the induction of cardiac injury in immunodeficient mice in vivo, supports the significance of direct virus-mediated cardiac injury in disease pathogenesis. Specific targeting of CVB in viral myocarditis will therefore, not only abrogate virus-mediated direct cardiac damage, but will also block immune response-mediated damage by blocking viral spread to uninfected tissue.

The host cell receptor for group B coxsackieviruses is the coxsackievirus-adenovirus receptor (CAR). This transmembrane protein is involved in the formation of tight junctions in the endothelium and in cell adhesion. Group B coxsackieviruses were shown to bind initially to the Decay Accelerating Factor (DAF) as a co-receptor, which activates intracellular signalling and transports the virus to the tight junctions, where it becomes internalized by CAR.

The CAR mediates cellular attachment for adenoviruses subtypes A and C-F and is essential to permissive infection of all 6 serotypes of CVB. CAR is a member of the immunoglobulin superfamily consisting of two extracellular Ig-like domains (D1 and D2), a transmembrane domain, and an intracellular tail of variable length. The N-terminal D1 domain has been shown to bind both adenovirus fiber knob protein and the canyon structure of CVB capsids.

Soluble decoy viral receptors have been found to efficiently inhibit the infection of rhino-, measles- and adenoviruses.

Various soluble variants of CAR (sCAR) have been detected, which originate by alternative splicing. Soluble (s) CAR proteins inhibit CVB infection of susceptible target cells in vitro and in vivo. The interaction of CVB3 with the sCAR leads to formation of altered (A) particles which are characterized by loss of VP4 from the virion shell and coincident irreversible loss of infectivity. It can be assumed that sCAR acts as a decoy and saturates epitopes on the virus surface that are essential for the interaction with the cellular receptor.

However, severe side effects with increased cardiac inflammation and heart injury have been observed following treatment of CVB3-infected mice with CAR4/7, a native sCAR variant with an intact CVB3-binding D1 domain, half of the D2 domain, and 23 amino acid long C-terminus.

Previous investigations have shown that dimeric sCAR, expressed as an immunoglobulin Fc-region fusion protein, has reduced systemic clearance and increased virus neutralizing capacity relative to monomeric sCAR and do not induce undesirable side effects.

To enhance solubility and extend half-life, the extracellular domain of CAR was fused to the Fc domain of human immunoglobulin G1 (IgG). Basically, sCAR-Fc proved to be a potent antiviral tool as it was suitable to protect cells and animals from CVB-3 infection. Under therapeutic conditions, however, when animals were treated with sCAR-Fc after CVB-3 infection, the antiviral efficiency decreased substantially.

Another promising new strategy for the inhibition of viruses is the application of RNA interference (RNAi). This evolutionary conserved mechanism of post-transcriptional gene silencing is triggered by double-stranded RNA molecules, which induce sequence-specific degradation of a target RNA.

In mammalian cells, double-stranded RNA molecules shorter than 30 nucleotides, known as small interfering RNAs (siRNAs), are usually employed to trigger RNAi without inducing an unspecific interferon response. The siRNAs become incorporated into a protein complex referred to as the RNA-induced silencing complex (RISC), in which the antisense strand of the siRNA acts as a guide to the target RNA, while the sense strand is degraded. After binding of activated RISC, cleavage of the target RNA by the Argonaut 2 protein is initiated. For the design of active siRNAs, thermodynamic features of the duplex as well as accessibility of the target region have to be taken into consideration.

Among other applications, RNAi has been found to efficiently inhibit viruses and clinical trials to treat infections with the respiratory syncitial virus, the human immunodeficiency virus and the Hepatitis B Virus have already been initiated. Successful application of RNAi for various enteroviruses was reported, including the inhibition of poliovirus, enterovirus 71, and CVB-3.

Just like for the sCAR-Fc approach, pre-treatment with RNAi efficiently protected cells from CVB-3 infections, but the antiviral activity was substantially lower when the curative approach was carried out with an ongoing CVB-3 infection.

### SUMMARY

In view of the above it is therefore one object of the invention to create an in vivo delivery system that would express a transgene, preferably a soluble receptor protein with high expression levels and without having undesirable side effects.

It is another object of the invention to provide a vector system and a composition for treating virally infected cells, especially cells infected with a virus of the Picornavirus family.

According to an exemplary embodiment of the invention the vector system comprises at least one viral vector and at least one regulable expression cassette inserted in said viral vector. The viral vector system facilitates a high and steady expression of the transgene. The regulable gene expression cassette governs the transgene expression. Simultaneously it provides the possibility of turning off the transgene expression in order to avoid potential side effects.

It is preferred that the at least one regulable expression cassette comprises at least one transactivator, at least one promoter and at least one nucleotide sequence coding for a transgene. Thus, all regulation systems are located on one single vector genome.

The cassette can be inducible, preferably by Doxycycline or any other known applicable inducer.

The expression cassette can be inserted into any region of the viral vector, preferably into the E-1 region of said vector.

The transactivator is preferably a second generation tetracycline-depended reverse transactivator (rtTA-M2) and the promoter a second generation tetracycline-depended response promoter (tight 1). The nucleotide sequence encodes preferably a soluble receptor protein or a part of it.

In an exemplary embodiment the vector system comprises two expression cassettes, whereby one expression cassette is regulated in a constitutive manner and/or a second expression cassette is regulated in an inducible manner.

It is also preferred that at least one expression cassette, especially the constitutive cassette, comprises at least one transactivator, preferably a second generation reverse tetracycline transactivator rtTA-M2, and at least one promoter, preferably a CMV promoter or a tissue specific promoter.

It is furthermore preferred that at least one expression cassette of the vector system, especially the inducible cassette, comprises at least one promoter, preferably a second generation tetracycline response promoter tight1, and at least one nucleotide sequence coding for a transgene, preferably for a soluble receptor protein or at least a part of a soluble receptor protein.

It is also possible to use instead of the components of the Tet-ON regulable system (rtTA-M2 and tight 1) the components of the Tet-OFF regulable system by using the tetracycline depending transactivator (tTA). It is also conceivable to use a transpressor as for instance the tetracycline transcriptional surpressor (tTs) instead of the transactivator.

The at least one transgene nucleotide sequence encodes for a soluble receptor protein or parts of it. The soluble receptor protein is preferably selected from a group comprising soluble Coxsackie-Adenovirus-receptor sCAR, rhinovirus receptor ICAM-1, human herpes virus receptor CD46, enterovirus receptor CD55, human poliovirus receptor, HIV receptor CD4 and HIV co-receptors CCR5 and CXCR4.

In one exemplary embodiment the transgene nucleotide sequence enodes for a fusion protein. The fusion protein comprises preferably a domain of the soluble receptor protein as the extracellular domain of the human soluble Coxsackie-Adenovirus-receptor (sCAR), rhinovirus receptor ICAM-1, human herpes virus receptor CD46, human poliovirus receptor, enterovirus receptor CD55, HIV receptor CD4 and HIV co-receptors CCR5 and CXCR4 and the Fc-domain of the human IgG1 or the C4b binding protein (C4 bp) α chain.

The regulable expression cassette is inserted into the vector either in tandem or in opposite direction.

In an exemplary embodiment the vector system comprises a first constitutive expression cassette comprising a CMV promoter and a second generation reverse tetracycline transactivator rtTA-M2 and a second inducible expression cassette comprising a second generation tetracycline response promoter tight1 and nucleotide sequence coding for a sCAR-Fc fusion protein according to sequence 1 or a sequence inverse to sequence 1.

The said transgene nucleotide sequence is advantageously codon optimized. This allows for a higher species specific transgene expression.

In one exemplary embodiment of the invention the translation and expression of the transgene is regulated by Doxycycline. It is also possible to induce expression of the transgene by addition of suitable antibiotics, nuclein acid molecules, as siRNA and other regulatory biomolecules.

The vector system is exemplary a non-leaky vector, i.e. the expression of transgene is either completely switched on in the presence of an inducer or completely switched off in the absence of an inducer.

After transduction of an organism with said vector and after induction the vector systems enables the expression of the transgene in a rate up to 500 ng in a ml blood plasma of an organism, preferably up to 700 ng/ml, preferably up to 1000 ng/ml, preferably up to 1500 ng/ml, preferably up to 2000 ng/ml, preferably up to 2500 ng/ml, preferably up to 2700 ng/ml, preferably up to 3000 ng/ml.

The vector system according to the invention is applicable as a medicament.

In one exemplary embodiment the vector system is applicable for treatment of cells infected with a virus of the Picornavirus family, especially in humans and newborn. The vector is preferably used for the treatment of meningitis, myocarditis, pancreatitis, hand, foot and mouth disease and Bornholm disease.

In an exemplary embodiment the vector system is applicable for treatment of CVB infected cells, preferably infected cardiac or pancreatic cells. The vector system can be used for in vitro and/or in vivo treatment of virally infected cells, preferably CVB infected cells, and most preferably CVB3 infected cells.

The vector system is also applicable for treatment of cells infected with adenovirus, especially cells infected with adenovirus A, C-F.

The treatment of viral infected cells, preferably infected cardiac or pancreatic cells, can also be carried out in combination with other viral inhibiting agents, preferably siRNA.

The applied siRNA is obtained synthetically or via expression from a vector, e.g. a plasmid or viral vector. siRNA can be expressed from a single vector. siRNA can also be expressed from the a vector comprising the nucleotide sequence of the siRNA and the soluble receptor protein.

It is most preferred to use a combination of the present viral vector with siRNA2 according to sequence 2 and siRNA4 according to sequence 3 for the treatment of infected cardiac cells.

The vector system is administered in vitro or in vivo before, simultaneously or after infection of the virally infected cells.

In an exemplary embodiment the infected cells are treated in vitro with an amount of the vector upto a MOI of 1 to 10, preferably upto a MOI of 3 to 8, most preferably upto a MOI of 5. Induction is preferably carried out with 1 to 1000 ng/ml doxycycline, preferably 100 to 800 ng/ml doxycycline, most preferably 500 ng/ml doxycycline.

In a further exemplary embodiment the vector dosage comprises 1×10¹⁰ to 1×10¹⁵ vector particles for in vivo, whereby for the treatment in mice 1×10¹⁰ to 3×10¹⁰ particles of viral vector are used and for in vivo treatment of human 10¹¹ to 10¹⁵, preferably 10¹³ vector particles are used.

The vector system is preferably based on a viral vector selected from the group comprising an adenoviral vector, a replication deficient adenoviral vector, an adeno-associated virus (AAV), a retrovirus vector, a reovirus vector, a herpes vector or a lentiviral vector having at least one deletion in at least one gene.

It is also of an exemplary advantage that a codon-optimized viral vector is used. It is possible to codon-optimize the CAP gene of the adeno-associated virus (AAV) in order to increase its expression and thus optimize the packaging.

In one exemplary embodiment the transgene, preferably a soluble protein is synthesized using a vector system having the above described features.

The viral vector system according to the invention enables the systemic release of a soluble receptor protein, preferably sCAR-Fc in the liver under the tight control of an inducible promoter. An adenoviral vector (AdV) was constructed that only expressed a soluble receptor protein, preferably sCAR-Fc in the presence of doxycycline (Dox). This vector is able to block viral infections, especially CVB3 infection in vitro and CVB3 infection and myocarditis in vivo, using haemodynamic and histological measurements to monitor cardiomyopathy post-CVB3 infection as shown in the examples.

Thus, therapeutic efficacy in treating CVB3 myocarditis with sCAR-Fc delivered from a pharmacologically regulated adenoviral vector is detectable. The treatment is highly efficient, without clinically observable side effects and leads to improved haemodynamics and heart function in CVB3-infected animals. Thus, combination of sCAR-Fc approach with gene therapeutic methods represents a new approach for treatment of cardiac CVB3 infections.

The object of the invention is also solved by providing a composition.

According to an exemplary embodiment of the invention the composition comprises a vector system having the above described features and antiviral siRNAs.

The applied siRNA is obtained synthetically or via expression from a vector, e.g. a plasmid or viral vector. siRNA can be expressed from a single vector. siRNA can also be expressed from the a vector comprising the nucleotide sequence of the siRNA and the soluble receptor protein.

The composition is applicable as a medicament.

In one exemplary embodiment the composition is applicable for treatment of cells infected with a virus of the Picornavirus family, especially in humans and newborn. The composition is preferably used for the treatment of meningitis, myocarditis, pancreatitis, hand, foot, mouth disease and Bornholm disease.

In an exemplary embodiment the composition is applicable for treatment of CVB infected cells, preferably infected cardiac or pancreatic cells. The composition can be used for in vitro and/or in vivo treatment of virally infected cells, preferably CVB infected cells, most preferably for CVB3 infected cells.

The composition is also applicable for treatment of cells infected with adenovirus; especially cells infected with adenovirus A, C-F.

In one exemplary embodiment the siRNA of the composition comprises siRNA2 comprising sequence 2 and siRNA4 comprising sequence 3.

It is preferred that the composition is administered to the cells before, simultaneously or after viral infection of the cells. The composition is advantageously applicable for the treatment of chronic infections of cardiac cells.

In one exemplary embodiment the composition comprises 1×10¹⁰ to 1×10¹⁵ particles of viral vector and 1 to 100 000 μg siRNA, preferably 100 to 10 000 μg siRNA, most preferably 500 to 5000 μg siRNA. For the in vivo treatment in mice 1×10¹⁰ to 3×10¹⁰ particles of viral vector are used and for in vivo treatment of human 10¹¹ to 10¹⁵, preferably 10¹³ vector particles are used.

The composition is exemplary obtained by mixing the viral vector and the siRNA.

The mixing is advantageously carried out immediately before administering the composition in vitro or in vivo. In this case the viral vector and the siRNA are stored separately before mixing. The viral vector is preferably stored in form of a solution comprising 1×10¹⁰ to 3×10¹⁵ particles of viral vector, preferably 10¹¹ to 10¹³ particles of viral vector. The siRNA is preferably stored in form of a solution comprising 1 to 100 000 μg siRNA, preferably 100 to 10 000 μg siRNA, most preferably 500 to 5000 μg siRNA.

If the siRNA is obtained by expression from a vector the vector is stored in solution or in a bacterial or viral host known to a person skilled in the art.

The object of the invention is also solved by a method of treating infections caused by a virus of the Picornavirus family, especially in humans and newborn, using a vector system with the above described features and/or a composition with the above described features.

The method is applicable preferably for treating meningitis, myocarditis and pancreatitis, hand, foot and mouth disease and Bornholm disease.

In an exemplary embodiment the method is applied for treatment of CVB infected cells, preferably infected cardiac or pancreatic cells. The method can be used for in vitro and/or in vivo treatment of virally infected cells, preferably CVB infected cells, most preferably for CVB3 infected cells.

The method is also applicable for treatment of cells infected with adenovirus, especially cells infected with adenovirus A, C-F.

In an exemplary embodiment of the invention the viral vector and the siRNA are administered separately. In this case the viral vector is administered in a concentration of 1×10¹⁰ to 1×10¹⁵ particles, whereby for the treatment in mice 1×10¹⁰ to 3×10¹⁰ particles of viral vector are used and for in vivo treatment of human 10¹¹ to 10¹⁵, preferably 10¹³ vector particles are used. siRNA is used in a concentration of 1 to 100 000 μg siRNA, preferably 100 to 10 000 μg siRNA, most preferably 500 to 5000 μg siRNA.

In another exemplary embodiment the viral vector and the siRNA are administered simultaneously. The applied concentrations are 1×10¹⁰ to 1×10¹⁵ vector particles for in vivo, whereby for the treatment in mice 1×10¹⁰ to 3×10¹⁰ particles of viral vector are used and for in vivo treatment of human 10¹¹ to 10¹⁵, preferably 10¹³ vector particles are used. siRNA is used in a concentration of 1 to 100 000 μg siRNA, preferably 100 to 10 000 μg siRNA, most preferably 500 to 5000 μg siRNA.

While both of the individual treatments, application of siRNAs and expression of a soluble receptor protein, especially in form of the fusion protein sCAR-Fc from the viral vector system only led to a moderate decrease of the viral load, the combination of both approaches in the composition according to the invention resulted in a strong synergistic antiviral effect. This is shown in the examples. Initially, a 4-log reduction of the virus titer was achieved, and even at the end of the experiment (day 13) the viral load was diminished by 3-logs, whereas the single treatments had completely lost their inhibitory activity.

Only the combination of treatment with the present viral vector, especially the viral vector AdG12, and siRNAs, especially siRNA against CVB-3 in form of the composition according to the invention is suitable to achieve both, an increase of cell viability and a substantial reduction of the virus titer. Both antiviral agents act in a synergistic manner. While sCAR-Fc expressed from the viral vector traps the virus extracellularly, siRNAs induce degradation of virus genomes that are present in the cells either at the beginning of the experiment or by entering the cells after circumventing the extracellular shield.

### BRIEF DESCRIPTION OF THE DRAWINGS

Examples of embodiments are explained in further detail by means of the following figures and examples.

FIGS. 1A-1B shows a structure of sCAR-Fc expressing adenoviral vectors and mechanism of sCAR-Fc mediated CVB3 inhibition.

FIGS. 2A-2C shows a Dox-dependent expression of sCAR-Fc by vector construct AdG12.

FIG. 3 shows an inhibition of ongoing CVB3 infection by sCAR-Fc.

FIGS. 4A-4D shows effects of AdG12 mediated pre-infectious sCAR-Fc expression on murine CVB3 myocarditis.

FIGS. 5A-5D shows AdG12 mediated therapeutic sCAR-Fc expression on murine CVB3 myocarditis.

FIG. 6 shows a relative CVB-3 titer of infected HMF cells in the lytic phase after treatment with siRNAs or sCAR-Fc.

FIG. 7 shows a relative CVB-3 titer in persistently infected HMF cells treated with siRNAs and/or sCAR-Fc.

FIG. 8 shows a virus titer of persistently CVB-3 infected HMF cells after repeated treatment with siRNAs and/or sCAR-Fc.

FIG. 9 shows a virus titer and cell viability of persistently infected HMF cells after two rounds of treatment

### DETAILED DESCRIPTION

### Materials and Methods

Coxsackievirus B3: In vitro and in vivo experiments utilized the genetically characterized, cardiovirulent Nancy strain of CVB3. Methods detailing virus propagation and titration of CVB3 in HeLa cells, as well as storage at −80° C., prior to infection of cells or animals were as previously described (Yanagawa B, Spiller O B, Proctor D G et al. Soluble recombinant coxsackievirus and adenovirus receptor abrogates coxsackievirus b3-mediated pancreatitis and myocarditis in mice. J Infect Dis 2004; 189:1431-9).

Cells: Human myocardial fibroblast (HMF) cell line (immortalized; HMF_{1226K/I}), HEK293 and HeLa cells (Wisconsin strain; courtesy of Dr. R. R. Ruckert, Madison) were propagated in monolayer culture in Minimal Essential Medium containing 5% heat inactivated fetal calf serum (FCS), 1% antibiotic/antimycotic, gentamycin and non-essential amino acids. Cell lines were propagated at 37° C. in a humidified atmosphere with 5% carbon dioxide.

Coxsackievirus infection of HMF cell line and cell viability assay: For the lytic infection assays, HMF cells were first transfected with siRNAs and/or transduced with AdG12 and inoculated with CVB-3 at a multiplicity of infection (m.o.i.) of 1 plaque forming unit (pfu) per cell in medium without FCS four hours thereafter for 30 minutes and maintained in cell culture medium. To generate persistently infected HMF cells, nearly confluent cells were inoculated with CVB-3 at an m.o.i. of 30. Medium was changed every other day. More than 90% of cells died within one week. Single cell clones grew up slowly and during the second week the cells were passaged. Henceforth, the infected cells were propagated by passaging twice a week in medium with a reduced FCS content of 2%. Virus titer in the supernatant was controlled regularly. After storage in liquid nitrogen and subsequent re-culturing, cells still produced high virus titer. For most of the experiments, the infected cells were seeded in 96-well (half area) plates and maintained for more than one week without passaging. As a measure of cytopathic effects induced by the CVB-3 in these non-subcultured HMF cells, cell viability was determined at several time points after treatment using the Cell Proliferation Kit II (Roche, Mannheim, Germany) according to the manufacturer's instructions. Measured absorbance at 492 nm thus correlates directly to cell viability.

Development of Adenoviral Vector, transduction and induction: sCAR-Fc was generated by fusion of the extracellular domain of human CAR with the carboxy terminus of human IgG1 Fc coding region. sCAR-Fc was cloned into the plasmid pZS2-CMV-rtTA downstream of the second-generation reverse tetracycline (tet)-dependent transactivator rtTA-M2 in two opposite directions. For generation of a Dox-regulated sCAR-Fc the Dox responsive tight1 promoter was inserted upstream of sCAR-Fc resulting in the adenoviral shuttle plasmids pAdG12-sCAR-Fc and pAdR4-sCAR-Fc. pAdG12-sCAR-Fc and pAdR4-sCAR-Fc were linearized with XbaI and ligated to the 5′ long arm of XbaI-digested E1-E3-adenovirus 5 mutant RR5. Transfection into HEK293 cells and propagation was carried out as described (Marienfeld U, Haack A, Thalheimer P et al. ‘Autoreplication’ of the vector genome in recombinant adenoviral vectors with different E1 region deletions and transgenes. Gene Ther 1999; 6:1101-13) generating the adenoviral vectors termed AdG12 and AdR4.

HMF cells were transduced with adenoviral vector at a concentration of 10 m.o.i. by addition of the required amount to the medium. Immediately after transduction the sCAR-Fc protein production was induced by adding Dox (1.5 μg/ml). Every second or third day Dox (and medium) was refreshed. When combined with siRNA double-transfections, AdG12 was transduced during the first and Dox was added after the second transfection.

siRNAs and transfection: siRNAs with two nucleotide overhangs used in this study were purchased from MWG Biotech (Ebersberg, Germany). Both, sRNA2 (target sequence CUA AGG ACC UAA CAA AGU U, Sequence 2) and siRNA4 (target sequence GUA CAG GGA UAA ACA UUA C, Sequence 3), are directed against the 3D RNA dependent RNA polymerase (3D^{pol}) of CVB-3 (GenBank acc. no. M33854; target nucleotides 6315-6333 and 6735-6753, respectively). As a control, an siRNA from Qiagen (Hilden, Germany) with no known homology in the human and viral genome was used. For transfection, HMF cells were seeded in 24-well plates at a density of 1.2×10⁵ cells per ml in a volume of 500 μl without antibiotics. The next day, cells were transfected with 12.5 nM siRNA 2 and 4 or 25 nM control siRNA and 2 μl Lipofectamine™ 2000 (Invitrogen, Karlsruhe, Germany) per well, following the manufacturer's instructions.

The persistently infected cells were plated in 96-well (half area) plates at a density of 10⁵ cells per ml in a volume of 50 μl. These cells were transfected twice on the same day with the siRNA concentrations denoted above using 0.125 μl Lipofectamine™ 2000. The supernatant was replaced by medium two hours after the first transfection and the second transfection mixture was left on the cells for about 20 hours.

Cell Cultures, Northern Blot, Western Blot, Virus Plaque Assays, IgG ELISA: HeLa (human cervical carcinomas) cells and HEK293 (human embryonal kidney) were cultured in Dulbecco's modified Eagle's medium (DMEM) (Gibco BRL, Karlsruhe, Germany) supplemented with 10% FCS and 1% penicillin/streptomycin. Northern and Western analysis and virus plaque assays were carried out as described (Fechner H, Pinkert S, Wang X et al. Coxsackievirus B3 and adenovirus infections of cardiac cells are efficiently inhibited by vector-mediated RNA interference targeting their common receptor. Gene Ther 2007; 14:960-71). Human IgG ELISA (Bethyl Laboratories Inc., Montgomery, Tex., USA) for detection of the Fc-tail of sCAR-Fc was performed according to supplier's introductions.

Determination of CVB-3 titer. The amount of infectious CVB-3 in the supernatant of infected HMF cells was determined on HeLa cells by an agar overlaid plaque assay as described. Shortly, the at least ten-fold diluted samples were incubated for 30 min on HeLa monolayers. Subsequently, cells were overlaid with agar containing Eagle's MEM. After incubation in a humidified atmosphere for two days, cells were stained with neutral red and virus titers were determined by plaque counting.

Detection of soluble CAR-Fc (SCAR-Fc). For analysis of sCAR-Fc expression, the supernatants of induced cultures were collected at different time points and stored at −20° C. sCAR-Fc protein levels were determined by the use of the Human IgG Enzyme Linked Immuno Sorbent Assay (ELISA) Quantitation Kit (Bethyl Laboratories, Montgomery, Tex., USA). Following the manufacturer's instructions, a MaxiSorb™ (Nunc, Langenselbod, Germany) 96 well plate was coated with a Goat anti-human IgG for one hour. During the blocking step the collected samples were diluted 1:10 and 100 μl were transferred to the reaction plate. After an additional incubation for one hour and an intensive washing, a Goat anti-human IgG-HRP conjugate in a 1:150.000 dilution was added to each well. Following the addition of a Tetramethyl Benzidine (TMB) substrate and a sulfuric acid, the oxidized product can be measured in a plate reader at 450 nm. As a calibrator, human reference serum in a working range of 3.9 ng/ml-500 ng/ml were used in each assay in duplicate. For calculation of results we used the calibrator as a standard curve with a four parameter logistic curve-fit.

Murine CVB3 myocarditis: AdG12 was injected into the vena jugularis of 6-8 weeks old Balb/c mice. Two days following AdV injection, mice were infected with 5×10⁴ pfu of CVB3 intraperitoneally. Dox (200 μg/ml) was orally administered to the mice via drinking water two days before CVB3 infection (preinfectious approach), concurrent or 1 d after CVB3 infection (therapeutic approach). Seven days post-CVB3 infection, the haemodynamic parameters of the mice were analysed as described (Fechner H, Sipo I, Westermann D et al. Cardiac-targeted RNA interference mediated by an AAV9 vector improves cardiac function in coxsackievirus B3 cardiomyopathy. J Mol Med 2008, 86:987-997), then blood was taken and organs were harvested for histopathological analysis. CVB3 positive-strand genomic RNA in tissues was detected by in situ hybridization using single-stranded ³⁵S-labeled RNA probes as described (Klingel K, Hohenadl C, Canu A et al. Ongoing enterovirus-induced myocarditis is associated with persistent heart muscle infection: Quantitative analysis of virus replication, tissue damage, and inflammation. PNAS 1992; 89:314-8) or standard plaque assay for CVB3 as described (Fechner H, Sipo I, Westermann D et al. Cardiac-targeted RNA interference mediated by an AAV9 vector improves cardiac function in coxsackievirus B3 cardiomyopathy. J Mol Med 2008, 86:987-997).

Statistics: Statistical analysis was performed by Student's t test (for data meeting parametric criteria) or Mann-Whitney U test (for non-parametric data analysis). Values are presented as the mean±the standard deviation, where n represents the number of independent experiments. Differences were considered significant at values of p<0.05.

### Example 1

### Doxycycline-Dependent Regulation of sCAR-Fc Expression

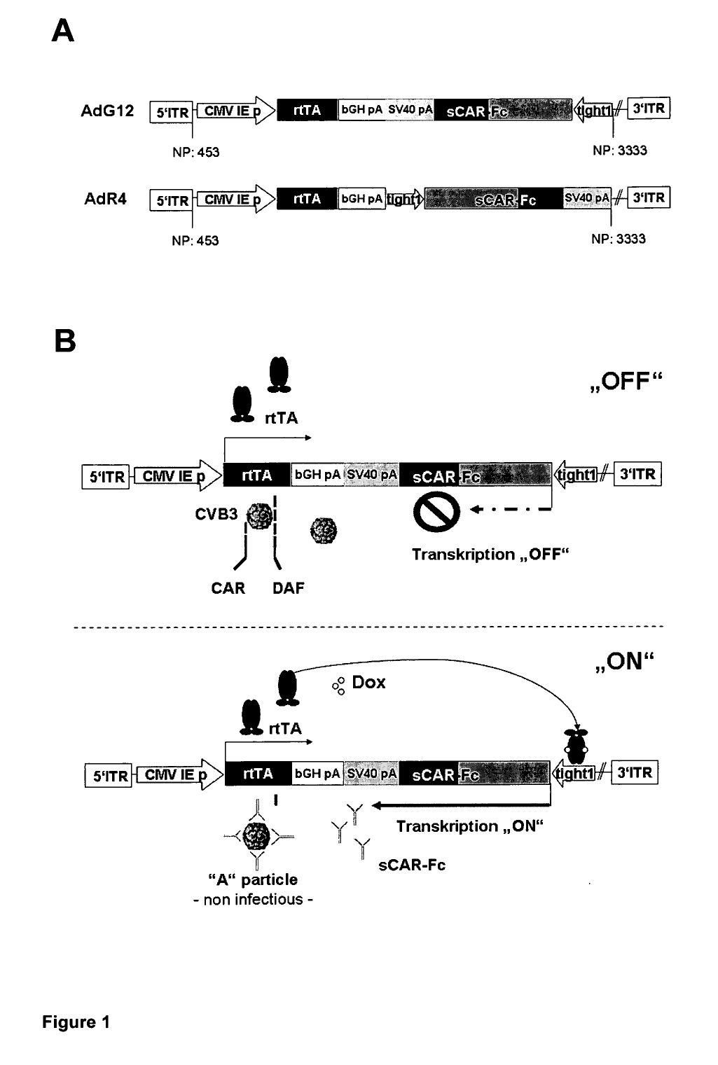

In order to achieve Doxycycline (Dox)-dependent sCAR-Fc expression two adenoviral vectors (AdV) were constructed. Each AdV contains two expression cassettes, one cassette for constitutive expression of the second generation reverse tetracycline transactivator rtTA-M2, the other for expression of sCAR-Fc from the improved second generation tetracycline (Tet) response promoter tight1. The expression cassettes were inserted either in tandem orientation (AdR4) or in opposite orientations (AdG12) into the E1 region of an E1-E3-adenovirus 5 backbone (see FIGS. 1A and 1B).

FIG. 1A is a schematic illustration of Dox-regulated sCAR-Fc expressing AdVs AdG12 and AdR4. Two expression cassettes, one for expression of the Dox-dependent transactivator rtTA-M2 and the other for Dox-inducible expression of sCAR-Fc were inserted into the E1 region between nucleotide position 453 and 3333 of an E1-E3-adenovirus 5 backbone. AdR4 contains the two expression cassettes in tandem direction, while in AdG12 the cassettes were inserted in opposite orientations.

FIG. 1B shows the mechanism of Dox-dependent adenoviral expression of sCAR-Fc and sCAR-Fc mediated inhibition of CVB3 Infection. In the absence of Dox the rtTA-M2 is unable to transactivate the tight1 promoter. Therefore, sCAR-Fc is not expressed and CVB3 infection cannot be inhibited (upper panel). In the presence of Dox rtTA-M2 transactivate the tight1 promoter, sCAR-Fc is expressed and interacts with CVB3 leading to formation of non-infectious A particles (lower panel). CMV IE p, immediate-early CMV promoter; rtTA, reverse tetracycline-controlled transactivator rtTA-M2; tight1: Dox-dependent response promoter; sCAR-Fc, fusion protein of the soluble extracellular domain of human CAR and the human IgG1 Fc region; SV40 pA and bGH pA, polyadenylation signal of SV40 and bovine growth hormone; 5′ITR, nucleotide positions 1-453 of adenovirus type 5 containing the left inverted terminal repeat of adenovirus 5 and the packaging signal Ψ; 3′ITR, right ITR of adenovirus 5.

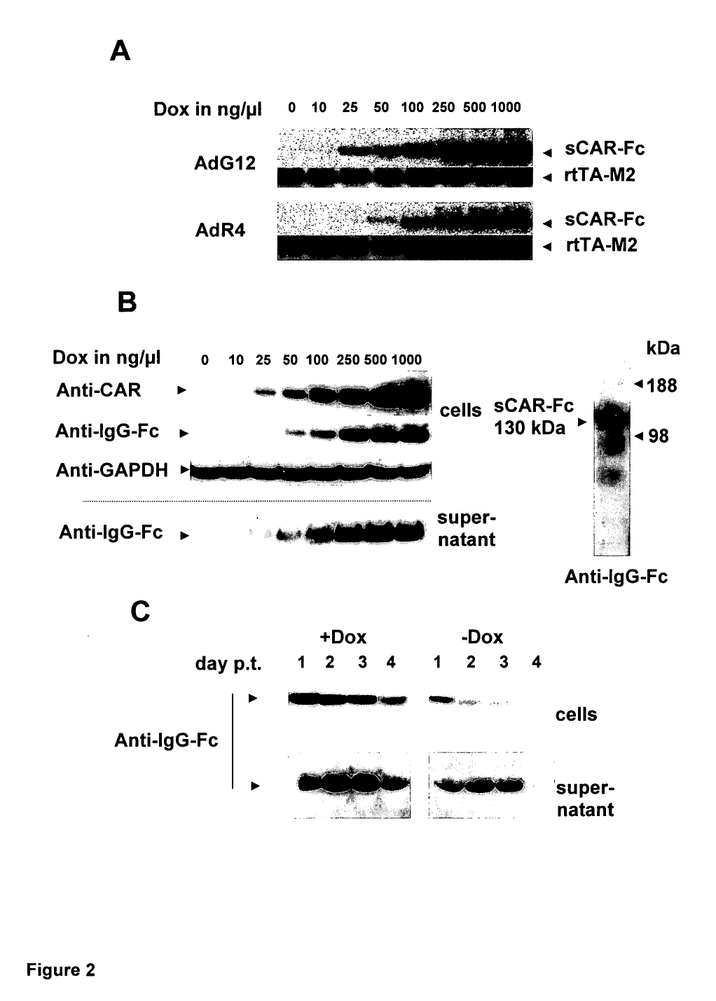

To analyse Dox-dependent regulation of AdV mediated sCAR-Fc expression, HeLa cells were transduced with AdR4 or AdG12 and cultured in the presence or absence of Dox. Northern blot analysis found sCAR-Fc mRNA expression to be strictly Dox-dependent, while rtTA-M2 expression was constitutively high (FIG. 2A). However, as detected by phosphoimaging of Northern-blots (FIG. 2A) Dox-induced sCAR-Fc mRNA expression was up to 6-fold higher in AdG12 compared to AdR4 transduced cells. Therefore, AdG12 was selected for further in vitro and in vivo studies.

sCAR-Fc protein was only detectable in AdG12 transduced cells and in the cell culture supernatant in the presence of Dox (FIG. 2B, left panel). As expected, Western blot analysis (under non-reducing conditions) confirmed that sCAR-Fc was expressed as a dimeric protein (FIG. 2B, right panel).

For safety reasons, non-leaky, induction confined expression of sCAR-Fc is an important feature of this gene therapy approach. As early as 24 h after transduction of HeLa cells with AdG12, in the presence of Dox, sCAR-Fc was detectable in the cell culture supernatant and achieved maximum extracellular concentration two to three days after transduction (FIG. 2C, left panel). Withdrawal of Dox after an initial 24 h induction period resulted in nearly complete loss of sCAR-Fc in the cell culture supernatant four days later (FIG. 2C, right panel). These results demonstrate rapid on/off switching of sCAR-Fc expression from AdG12.

FIG. 2A shows the expression of sCAR-Fc mRNA. HeLa cells were transduced with AdG12 and AdR4, each at a MOI of 2, and then cultured in the presence and absence of Dox. Northern blot analysis performed 48 h after transduction showed Dox-dose dependent increase of sCAR-Fc mRNA expression for both vectors, while rtTA-M2 expression stayed constant. sCAR-Fc transcription could not be detected in the absence of Dox.

FIG. 2B shows the expression of sCAR-Fc protein. HeLa cells were transduced with AdG12, and sCAR-Fc expression was induced as described in (A) above. sCAR-Fc was detected by Western analysis (reducing conditions) in both cells and cell culture supernatant using antibodies directed against human CAR and human IgG-Fc domain. Immunoreactivity against GAPDH was used as loading control (left panel). Right panel: Dimeric sCAR-Fc detected by western blotting under non-reducing conditions in cell culture supernatant.

FIG. 2C shows the On/off switching mode of sCAR-Fc expression. HeLa cells were transduced with AdG12 at a MOI of 2 and incubated with Dox (1 μg/ml). After 24 h (day 0) medium was replaced by fresh medium and cells were cultured for an additional 4 days with Dox (left panel) or without Dox (right panel) During this time, medium was replaced daily with fresh medium. sCAR-Fc was detected in both cells and medium by Western analysis using an anti-IgG-Fc antibody.

### Example 2

### Inhibition of CVB3 Infection by sCAR-Fc Vector In Vitro

Next the sCAR-Fc mediated inhibition of CVB3 in vitro as a function of AdG12 dose, Dox concentration and the dose of CVB3 was studied. Transduction of HeLa cells with 5 MOI of AdG12 and induction with 500 ng/ml Dox for 48 h were sufficient to prevent CVB3 infection in sCAR-Fc expressing cells completely. Under these tranductional conditions sCAR-Fc expression levels reached a maximum of 29.4 μg/ml in the cell culture supernatant. sCAR-Fc expressed from AdG12 could efficiently block CVB3 doses of up to 2.5 MOI (data not shown). Therefore, sCAR-Fc expressed by AdG12-transduced cells efficiently inhibited CVB3 infection of these cells and secreted sCAR-Fc levels were directly related to initial AdG12 MOI and Dox concentration.

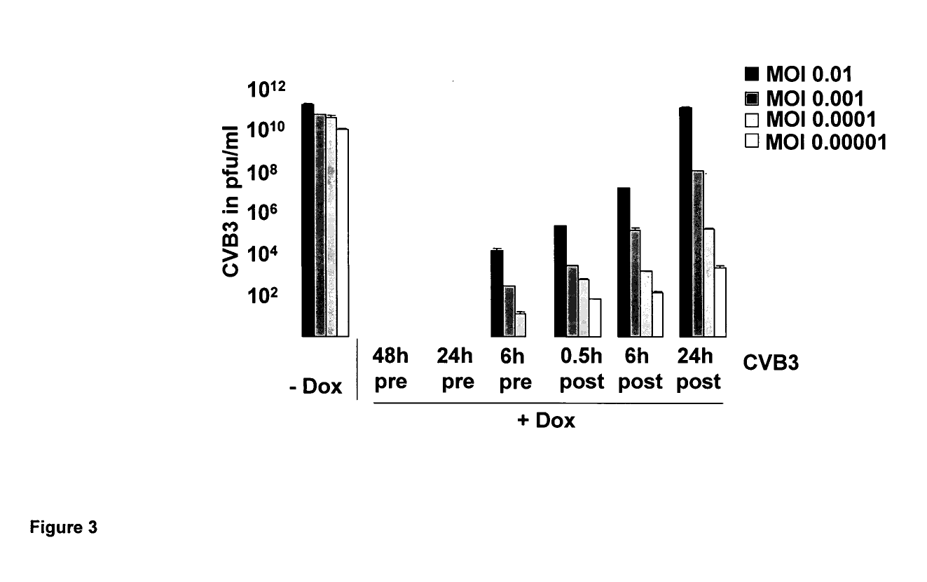

To assess the potential of AdG12 to suppress ongoing infections as this represents the typical situation encountered the clinical setting, HeLa cells were transduced with AdG12 and sCAR-Fc expression was induced with Dox at different times relative to CVB3 infection. Expression of sCAR-Fc 48 h and 24 h before CVB3 infection resulted in complete inhibition of CVB3 infection in the transduced cells. The inhibitory efficiency of sCAR-Fc was gradually reduced the later the sCAR-Fc expression was induced. However, even if sCAR-Fc expression was induced 24 h after infection CVB3 progeny virus number was still reduced by about 10⁶-fold compared to controls without sCAR-Fc (FIG. 3) demonstrating high efficacy of sCAR-Fc in ongoing CVB3 infections.

FIG. 3 shows the inhibition of ongoing CVB3 infection by sCAR-Fc. HeLa cells were transduced with AdG12 at a MOI of 5 and infected with CVB3 48 h later as described in FIG. 3A. CVB3 replication was analysed by plaque assays after 48 h of culture. For induction of sCAR-Fc expression Dox (1 μg/ml) was added to the medium at the points of time indicated, from 48 h before to 24 h after CVB3 infection.

### Example 3

### Systemic sCAR-Fc Gene Transfer Supports Inducible sCAR-Fc Delivery In Vivo

To examine the effect of AdG12 mediated sCAR-Fc expression on the progression of CVB3-induced myocarditis first sCAR-Fc expression kinetics following intravenous administration of 3×10¹⁰ particles of AdG12 to Balb/c mice was determined. sCAR-Fc serum concentrations in AdG12 (+Dox) transduced animals roughly doubled from day 2 (254±29 ng/ml) to day 5 (464±159 ng/ml), then decreased at day 8 (147±60 ng/ml), but expression did not decrease further when measured at day 14 (141±51 ng/ml). In the absence of Dox, sCAR-Fc serum levels were indistinguishable from levels in untransduced control mice (data not shown). Histopathological examination of liver and heart samples did not show any signs of tissue damage and inflammation at various time points (not shown).

### Example 4

### Preinfectious sCAR-Fc Gene Therapy Prevents Cardiac Dysfunction and Inflammation

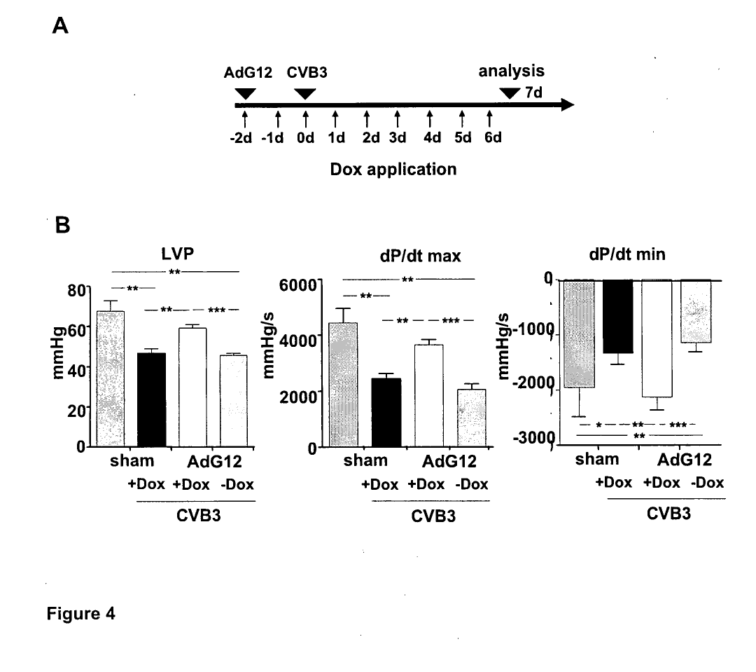

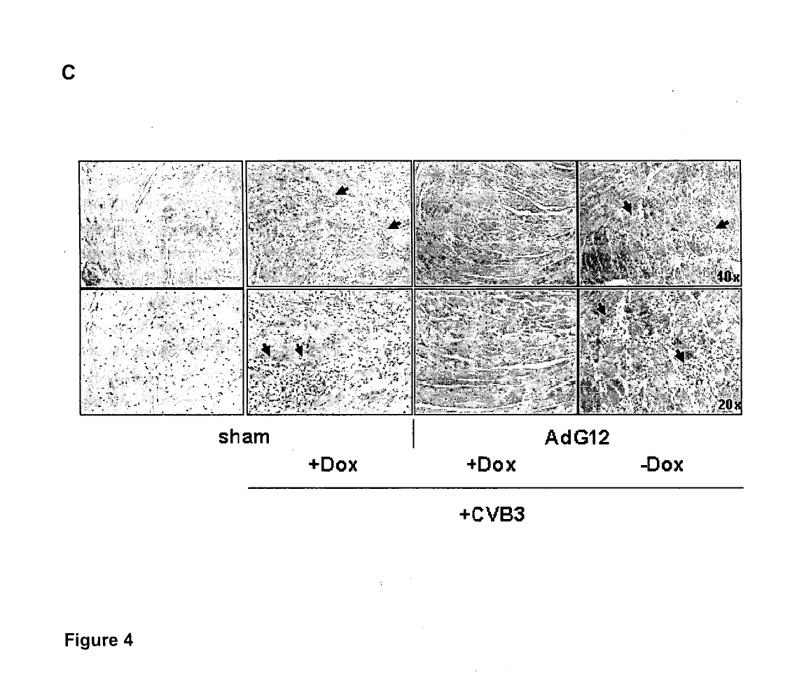

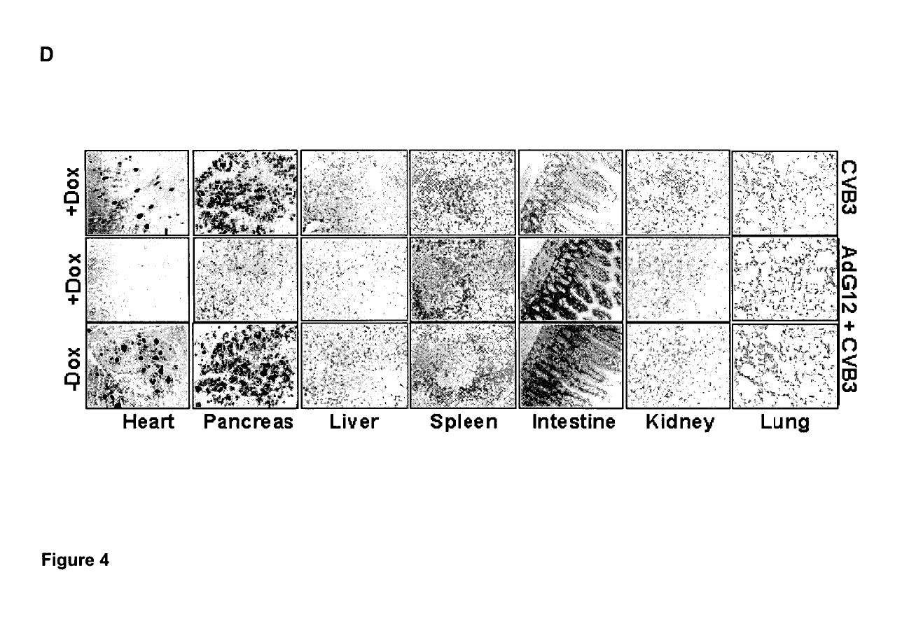

Based on sCAR-Fc in vivo expression kinetics determined above, the ability of AdG12 transduced mice to inhibit CVB3-mediated myocarditis was performed. Mice were transduced with AdG12 and sCAR-Fc expression induced and maintained via permanent oral Dox administration. The AdG12 doses was reduced to 1×10¹⁰ virus particles per mouse as in the initial experiment with 3×10¹⁰ particles AdG12 the sCAR-Fc serum levels distinctly exceeded therapeutical relevant levels that were already found below 100 ng/ml. Two days after vector transduction animals were infected with 5×10⁴ pfu CVB3 (FIG. 4A). At the point of CVB3 infection, circulating sCAR-Fc concentrations were 228.5±174 ng/ml and seven days later when mice were sacrified and analysed sCAR-Fc concentrations were 99.63±22.7 ng/ml. No sCAR-Fc was measured in the AdG12-transduced mice that did not receive Dox, which were identical to animals that did not receive AdG12. No mortality was observed in any of the groups. CVB3-infected mice that did not receive AdG12 or were transduced with AdG12 in the absence of Dox administration showed a continuous loss in body weight, resulting in an average 30% decrease by day 7 post-infection. By comparison, CVB3-infected mice that received AdG12 and Dox only lost roughly 5% of their body weight (data not shown). Haemodynamics were measured by tip catheter on day seven after CVB3 infection. Animals with CVB3 myocarditis showed disturbed left ventricular (LV) function with impaired parameters of contractility (dP/dtmax 2428±490 vs. 4429.3±1287 mmHg/s, p<0.01; LVP 46.6±6 vs. 67.5±13 mmHg/s, p<0.01) and diastolic relaxation (dP/dtmin−1330.5±437 vs. −1950±910 mmHg/s, p<0.05) as compared with non-infected control mice. AdG12 (+Dox) treated CVB3-infected mice had significantly improved cardiac contractility and diastolic relaxation compared with CVB3 infected animals transduced with AdG12 in the absence of Dox (dP/dtmax 3645.1±443 vs. 2057.9±490 mmHg/s, p<0.001; LVP 59±4 vs. 45.4±3 mmHg/s, p<0.001; dP/dtmin −2125.5±282 vs. −1143.6±246 mmHg/s, p<0.001) and CVB3 infected control mice (dP/dtmax 3645.1±443 vs. 2428±490 mmHg/s, p<0.01; LVP 59±4 vs. 46.6±6 mmHg/s, p<0.01; dP/dtmin −2125.5±282 vs. −1330.5±437 mmHg/s, p<0.01), respectively. Importantly, haemodynamics of CVB3 infected animals treated with AdG12 (+Dox) were similar to non-infected control animals (FIG. 4B). Heart section samples revealed extensive areas of damage with myocyte necrosis and infiltration of mononuclear cells in CVB infected control mice as well as in CVB3-infected AdG12 (−Dox) mice (myocarditis score of both groups 3-4). Cell damage and inflammation was completely absent in AdG12 (+Dox) group (myocarditis score=0) and showed histology comparable to hearts of sham-infected mice (FIG. 4C).

FIG. 4A shows the application scheme and timeline of sample preparation. Mice were transduced with 1×10¹⁰ particles of AdG12 (n=12) and sCAR-Fc expression was induced and maintained through Dox in 6 of the 12 animals. Twelve control mice were sham operated and 6 of them treated with Dox. AdG12 (+Dox), AdG12 (−Dox) and sham operated (+Dox) animals were infected with 5×10⁴ pfu of CVB3 two days later and analysed seven days after CVB3 infection.

FIG. 4B shows the effect of sCAR-Fc and CVB3 infection on cardiac function. The left ventricular function in CVB3 infected animals was severely disturbed with impaired contractility (LVP, dP/dtmax) and relaxation (dP/dtmin) when compared to sham operated controls without CVB3 infection. AdG12 transduced animals with sCAR-Fc expression (AdG12 (+Dox)) had significantly improved systolic and diastolic LV function when compared to AdG12 transduced animals without sCAR-expression (AdG12 (−Dox)) or to CVB3 infected sham operated mice with Dox treatment. *p<0.05; **p<0.01, ***p<0.001. Values are given as mean values±S.E.M.

FIG. 4 C shows the prevention of CVB3 induced heart injury through AdG12. Upper panel: 10-fold magnification. Lower panel: 20-fold magnification. Heart sections were stained with haematoxylin & eosin (H&E). sCAR-Fc expressing animals (AdG12 (+Dox)) exhibit complete preservation of myocardial integrity similar as observed in sham operated control animals, while in AdG12 (−Dox) and sham operated (+Dox) control animals, extensive areas with myocyte necrosis and inflammation were prominent. Arrows represent extensive areas of inflammation.

### Example 5

### Gene Therapy Inhibits CVB3 Infection of Heart and Pancreas

To document whether absence of pathological changes of the heart correlates with cardiac CVB3 infection we performed radioactive in situ hybridization experiments to visualize the presence of plus strand CVB3 RNA at the cellular level at a high sensitivity. Animals of AdG12 (+Dox) CVB3 infected group did not show any CVB3-infected cells in the heart (FIG. 4D). Moreover, individuals of this group showed no (FIG. 4D) or minimal (results not shown) levels of CVB3 RNA in the pancreas, which is the primary site of CVB replication and most susceptible organ for CVB infection in mice. In contrast, CVB3-infected and AdG12 (−Dox) CVB3-infected mice showed high prevalence of CVB3 RNA in the heart and pancreas. In other organs (spleen, liver, kidney, intestine and lung) CVB3 RNA was undetectable in AdG12 (+Dox) as well as in the control groups by in situ hybridization (FIG. 4D). Thus, sCAR-Fc efficiently protected mice from virus entry and subsequent from replication in the heart and other organs.

FIG. 4D shows the virus entry and replication into the heart and pancreas is blocked by sCAR-Fc. The distribution of viral RNA was visualized by in situ hybridization using a ³⁵S-labeled RNA probe specific to CVB3. In CVB3 infected sham operated (+Dox) control mice heart cardiomyocytes are infected as indicated by the black precipitate representing the virus RNA. CVB3 infection was also detected in pancreas, while spleen, lung, gut, kidney and liver were not infected. No virus-positive cell could be detected in AdG12 (+Dox) mice in all of the organs investigated while in AdG12 (−Dox) a similar organ distribution of CVB3 infection as in CVB3 infected sham operated (+Dox) control mice was observed.

### Example 6

### sCAR-Fc Gene Therapy Improves Cardiac Contractility and Reduced Cardiac Demaging in Pre-Excisting CVB3 Infection

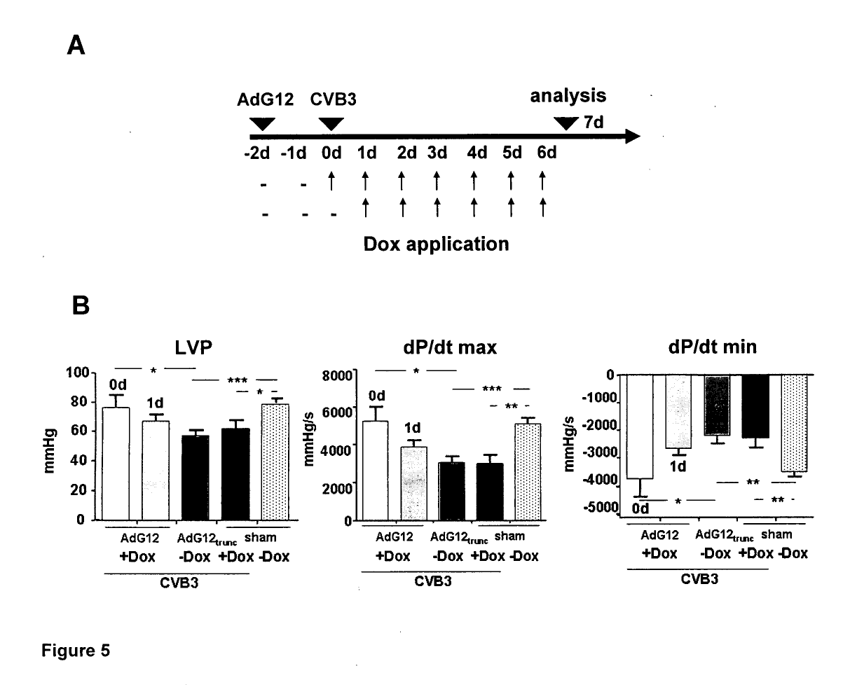

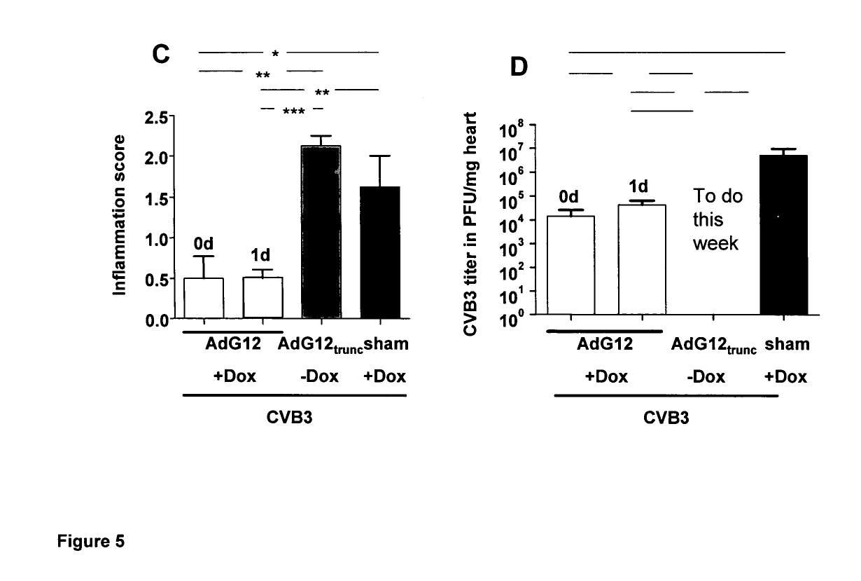

In a next step the efficacy of sCAR-Fc gene therapy in a therapeutic approach was analyzed. Mice were transduced with AdG12 and Dox was applied for induction of sCAR-Fc either concurrent with CVB3 infection two days after transduction or one day after CVB3 infection at day three after transduction (FIG. 5A). Accordingly to the experimentally procedure of this approach sCAR-Fc was undetectable two days after AdG12 induction but showed serum levels of 28.4 ng/ml already 16 h after induction with Dox (data not shown). Compared to sham operated untreated controls body weight was reduced about 16% in animals with concurrent induction of sCAR-Fc, while in the group with sCAR-Fc expression induced one days after CVB3 infection body weight loss about 25, which in fact was close similar to CVB3 infected untreated control groups (data not shown). Compared to CVB3 infected animals transduced with the control vector AdG12_{trunc}, which do not expresses sCAR-Fc induction of sCAR-Fc concurrent with CVB3 led to significantly improved cardiac contractility and diastolic relaxation (dP/dtmax 5214±798.8 vs. 3012±347.1 mmHg/s, p<0.02; LVP 76.4±8.6 vs. 56.8±3.9 mmHg/s, p<0.05; dP/dtmin −3757±634.2 vs. −2212±281.8 mmHg/s, p<0.05), which in fact were in the range on uninfected animals. In contrast, Animals with induced sCAR-Fc expression after CVB3 infection did not show improved haemodynamic parameters compared to the AdG12_{trunc} group (FIG. 5B). Heart section samples revealed reduced myocarditis score of both, concurrent and post infection sCAR-Fc treatment groups (myocarditis score=0.5 vs. 2 and 1.5, respectively) and strong reduced titers of infectious CVB3 (>2 log₁₀ steps) in the heart compared to CVB3 infected control animals (FIG. 5C,D). In situ hybridization confirms reduced presence of CVB3 RNA in the heart of sCAR-Fc expressing animals.

FIG. 5A shows an application scheme and timeline of sample preparation. Mice were transduced with 1×10¹⁰ particles of AdG12 (n=12) and animals infected with CVB3 two days later. sCAR-Fc expression was induced through Dox in 5 animals concurrent (AdG12+Dox; 0 d) with and in 7 animals (AdG12+Dox; 1 d) one day after CVB3 infection, respectively. Seven mice were transduced with 1×10¹⁰ particles of the control adenoviral vector (AdG12_{trunc}−Dox) which has sequence identity to AdG12 but do not express sCAR-Fc (not shown) and infected with CVB3 two days after transduction. Eleven mice were sham operated and four of them treated with Dox and infected with CVB3 (sham+Dox).

FIG. 5B shows the effect of sCAR-Fc and CVB3 infection on cardiac function. The left ventricular function in CVB3 infected non treated animals was severely disturbed with impaired contractility (LVP, dP/dtmax) and relaxation (dP/dtmin) when compared to sham operated controls without CVB3 infection. AdG12 transduced animals with sCAR-Fc expression (AdG12+Dox, 0 d) had significantly improved systolic and diastolic LV function when compared to control vector AdG12_{trunc}−Dox transduced animals. *p<0.05; **p<0.01, ***p<0.001. Values are given as mean values±S.E,M.

FIG. 5C shows the Myocarditis score of CVB3 infected and sCAR-Fc treated groups. For description of groups see FIG. 5A. Shown are mean values±S.E.M. *p<0.05; **p<0.01; ***p<0.001.

FIG. 5D shows the infective virions in the heart. Cardiac tissue samples were homogenized, and viral titers were assessed by plaque assay. For description of groups see FIG. 5A. Shown are mean values±S.E.M. *p<0.05; **p<0.01; ***p<0.001.

### Example 7

### Pre-Incubation of Uninfected HMF Cells with siRNAs and/or sCAR-FC Followed by Infection of HMF Cells with CVB-3

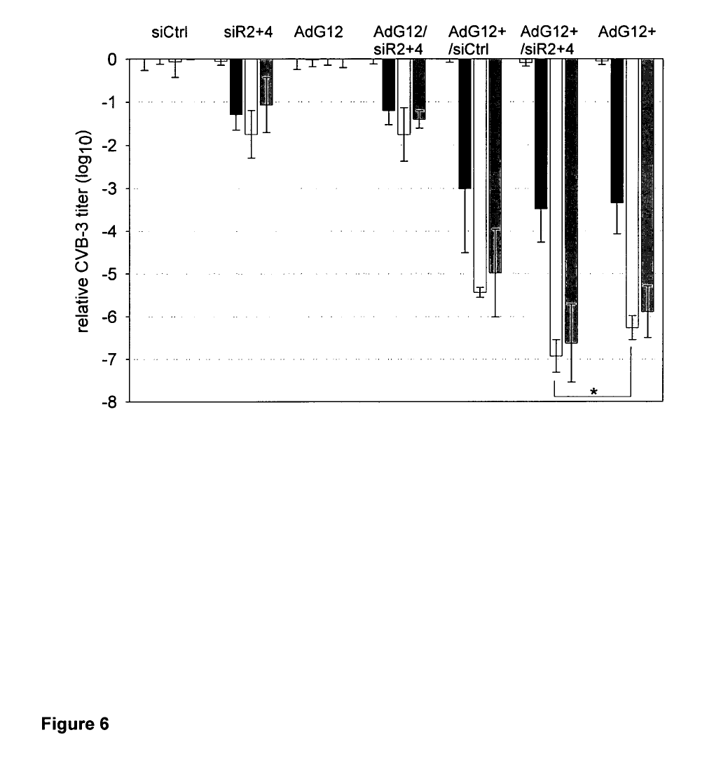

For a first assessment of the antiviral potential of both strategies, uninfected human myocardial fibroblasts (HMF) were initially pre-incubated with 12.5 nM of each of the siRNAs 2 and 4, both of which are directed against the viral 3D^{Pol}, and inoculated with 1 m.o.i. of CVB-3 four hours thereafter. Virus titer on subsequent days was determined by titration of culture supernatants on confluent HeLa cells. A reduction of more than 1-log was observed after 24 hours and lasted for at least three days (FIG. 6). As expected, transduction with the doxycycline-(Dox-) inducible sCAR-Fc expressing adenoviral vector AdG12 did not affect the virus titer in the absence of Dox. Induction of the sCAR-Fc expression by the addition of Dox to AdG12 transduced cells resulted in a 3-log decrease of CVB-3 titer on the first day and up to 6-log lower virus titers on days two and three after infection.

The combination of sCAR-Fc expressing adenoviral vector and antiviral siRNAs yielded an additive increase of the inhibitory activities resulting in an almost 7-log reduction of the virus titer. Closer statistical analysis revealed that the antiviral activity of the combination of sCAR-Fc and siRNAs was significantly higher than the inhibitory effect of the sCAR-Fc expressing vector in the presence of a control siRNA on day 2 of the experiment.

FIG. 6 shows the relative CVB-3 titer of infected HMF cells in the lytic phase after treatment with siRNAs or sCAR-Fc. Cells were transfected with 12.5 nM of each siRNA and/or transduced with AdG12 at an m.o.i. of 10 with (+) or without addition of Dox. Infection with CVB-3 at an m.o.i of 1 was carried out four hours thereafter. The supernatants were collected one hour (light grey), 1 day (black), 2 days (white) and 3 days (dark grey) after infection with CVB-3 and virus titers were determined on HeLa cells. Mean values±SD of three independent experiments each performed in duplicate are shown. siCtrl: control siRNA; siR2+4: siRNA 2 and 4 against 3D^{pol} of CVB-3; AdG12: adenoviral vector expressing sCAR-Fc. *p<0.05

### Example 8

### Treatment of HMF Cells with an Ongoing CVB-3 Infection with siRNAs and/or sCAR-FC

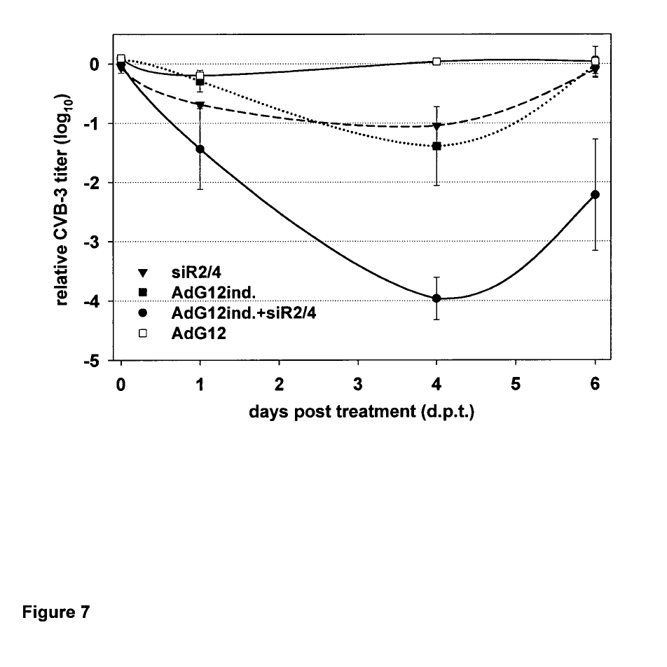

In the next step, the antiviral potential of both siRNAs and AdG12 in HMF cells with an ongoing CVB-3 infection was tested. For this purpose, the persistently infected cells were transfected with siRNAs 2 and 4 twice a day on two consecutive days. After the treatment, the virus titer decreased by 1-log(FIG. 7). Comparable results were obtained with sCAR-Fc expressed from AdG12. In contrast to the lytic infection assays described above, combination of both treatments (siRNAs plus sCAR-Fc) led not only to a slight additive increase of antiviral activity, but rather enhanced virus inhibition to give a 4-log reduction of virus proliferation in persistently infected HMF cells. As can be seen in FIG. 7, the most pronounced virus inhibition was obtained on day four after the treatment, but the antiviral effect was drastically diminished by the end of the week. A possible explanation for this finding is the transient nature of siRNA-mediated silencing as well as temporally restricted production of sCAR-Fc from the adenoviral vector.

FIG. 7 shows the relative CVB-3 titer in persistently infected HMF cells treated with siRNAs and/or sCAR-Fc. Cultures were transfected with 12.5 nM siRNAs 2 and 4 on two consecutive days (triangle) or transduced with AdG12 (open square: in the absence of Dox; filled square: in the presence of Dox) or both (siRNA 2 and 4 plus AdG12 in the presence of Dox (filled circle)). Virus titer of the collected supernatants was determined on HeLa cells. Shown are mean values±SD of six independent experiments, each performed in duplicate.

To investigate the time course of sCAR-Fc expression in persistently infected HMF, the amount of protein in the supernatant was quantified by a human IgG ELISA. For these quantifications, supernatants of cells transduced with AdG12 and induced by the addition of Dox were collected and measured (data not shown). The amount of detected protein corresponded to approximately 10 to 100 ng Fc-domains per ml supernatant. The protein level dropped drastically after day 4 of the experiment and could be restored by a second transduction with the AdG12 on day 6. Initially, hardly any difference was observed between cells, which were only transduced with AdG12 in the presence of Dox, and cells, which underwent additional treatment with siRNAs 2 and 4 (data not shown). Owing to the improved cell viability, higher sCAR-Fc levels were detected for the double-treated cells at later time points. Taken together, the time course of secreted sCAR-Fc levels in the supernatant was comparable in both types of experiments and prolonged high-level expression of sCAR-Fc can be achieved by a second transduction of the cells.

### Example 9

### Repeated Treatment of HMF Cells with an Ongoing CVB-3 Infection with siRNAs and/or sCAR-FC

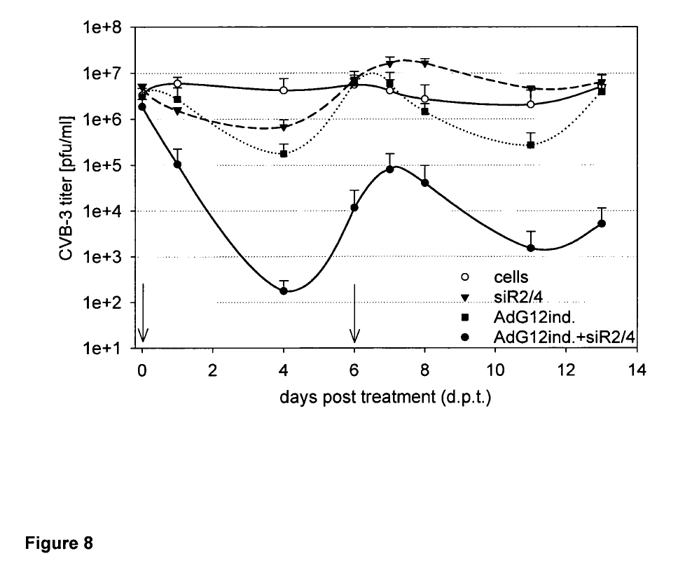

In order to compensate for the loss of antiviral impact the HMF were transfected and/or transduced cells again on day six of the experiment. As can be seen in FIG. 8, a second treatment with siRNAs directed against the virus did not restore a substantial antiviral effect. In contrast, the additional transduction of the cells with AdG12 inhibited virus replication again and led to a 1-log reduction of CVB-3 titer on day eleven of the experiment. For the double-treatment approach with both, siRNAs and sCAR-Fc, the titer initially decreased from about 5×10⁶ to 10² pfu/ml corresponding to a 4.5-log reduction and then rose to 10⁵ pfu/ml on day 7 of the experiment. The titer was reduced to approximately 10³ pfu/ml again after the second round of treatment, which corresponds to a 3.5-log inhibition of the virus.

According to these results the combination strategy with siRNAs and AdG12 is considerably more efficient in inhibiting CVB-3 in persistently infected HMF cells than either of the single approaches. Furthermore, repeated treatments are required to maintain inhibition.

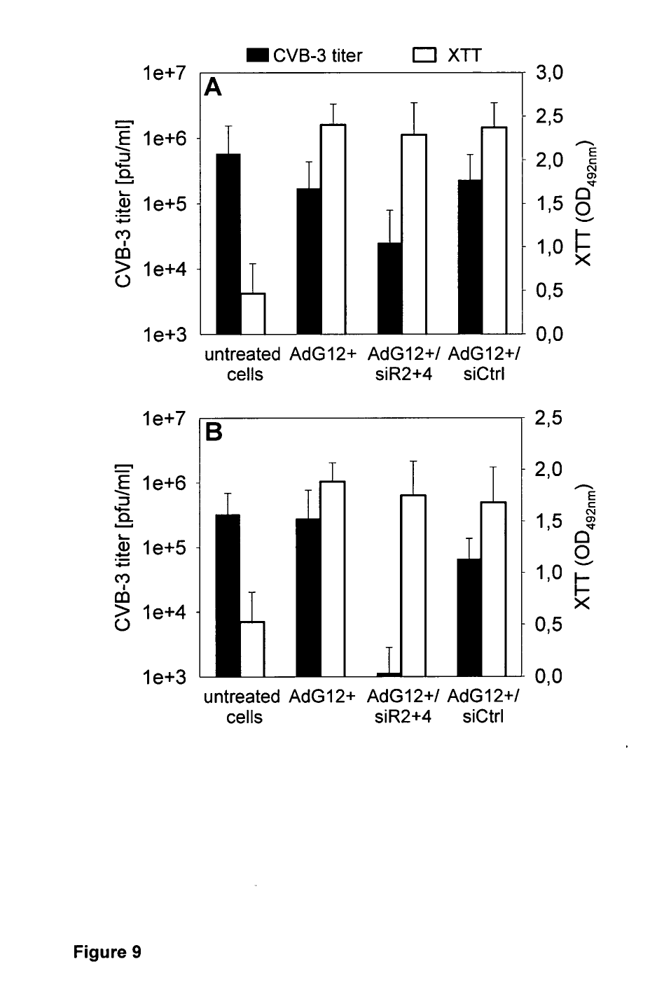

In an next step the question was tackled whether it will be necessary to repeat the double treatment or if it might be sufficient only to use the adenoviral vector for the second administration. To address this question, persistently infected HMF cells were initially treated with both siRNAs and AdG12. Cultures that do not undergo a second round of treatment loose viability (FIG. 9, white bar). The viability loos is concomitant with a high virus titer (black bar) at day eight of the experiment. When cells were transduced with AdG12 at this time point, cell viability was high as measured by XTT assays. However, despite the protective effect of sCAR-Fc against cell lysis, the virus titer remained comparatively high after the second application of the virus vector. In contrast, combination treatment with AdG12 and siRNAs not only maintained high cell viability, but also substantially reduced the virus titer. A reduction of the virus titer by approximately 1-log was observed on day 8 (FIG. 9A), and the effect increased to an approximately 2.5-log inhibition at day 11 of the experiment (FIG. 9B), indicating the beneficial outcome of the double treatment.

FIG. 8 shows the virus titer of persistently CVB-3 infected HMF cells after repeated treatment with siRNAs and/or sCAR-Fc. Cultures were transfected and/or transduced on day 0 and 6 of the experiment (arrows). Cells were either transfected with 12.5 nM siRNAs 2 and 4 (triangles), transduced with AdG12 (filled square), or simultaneously transfected with siRNAs 2 and 4 and transduced with AdG12 (filled cirlces). Titer of untreated cells is shown as a control (open circles). For the induction of sCAR-Fc expression from AdG12, Dox was added to the medium. Virus titers in the supernatant were determined on HeLa cells. Mean values and standard deviations of three independent experiments, each performed in duplicate, are shown.

FIG. 9 shows the virus and cell viability of persistently infected HMF cells after two rounds of treatment. Initially, cells were transduced with AdG12 and transfected with siRNAs 2 and 4. Six days after the first treatment cells were transduced with AdG12 again, either without siRNA transfection or in combination with siRNAs (2 and 4) and control siRNA, respectively as indicated. Both virus titer in the supernatants and cells were analysed at day 8 (A) and day 11 (B) after the first treatment. Virus titer (black bars) of the supernatant was determined on HeLa cells. XTT absorbance measured at 492 nm (white bars) correlates directly with cell viability. Untreated cells were neither treated during the first nor the second round. Shown are mean values and standard deviations of five independent experiments each performed in duplicate. siCtrl: control siRNA; siR2+4: siRNA 2 and 4 against 3D^{pol} of CVB-3; AdG12: adenoviral vector expressing sCAR-Fc; the ‘+’ symbol denotes addition of doxycyclin.

1-35. (canceled)

36. A vector system comprising at least one viral vector and at least one regulable expression cassette inserted in said viral vector.

37. The vector system according to claim 36, wherein the at least one regulable expression cassette comprises at least one transactivator, at least one promoter and at least one nucleotide sequence coding for a transgene.

38. The vector system according to claim 36, wherein the at least one regulable expression cassette is inserted into any region of said vector, preferably into the E-1 region of said viral vector.

39. The vector system according to claim 36, wherein the cassette is inducible, preferably by Doxycycline.

40. The vector system according to claim 36, wherein said vector comprises two expression cassettes.

41. The vector system according to claim 36, wherein the at least one expression cassette comprises at least one transactivator, preferably a second generation reverse tetracycline transactivator rtTA-M2, and a promoter, preferably a CMV promoter or a tissue specific promoter.

42. The vector system according to claim 36, wherein the at least one expression cassette comprises at least one promoter, preferably a second generation tetracycline response promoter tight1, and at least one nucleotide sequence coding for a transgene, preferably for a soluble receptor protein or at least a part of a soluble receptor protein.

43. The vector system according to claim 36, wherein the at least one transgene nucleotide sequence encodes for a soluble receptor protein or at least a part of a soluble receptor protein or fusion protein.

44. The vector system according to claim 43, wherein the fusion protein comprises the extracellular domain of the human soluble Coxsackie-Adenovirus-receptor (sCAR), rhinovirus receptor ICAM-1, human herpes virus receptor CD46, human poliovirus receptor, enterovirus receptor CD55, HIV receptor CD4 and HIV co-receptors CCR5 and CXCR4 and the Fc-domain of the human IgG1 or the C4b binding protein (C4 bp) α chain.

45. The vector system according to claim 42, wherein the translation and expression of the transgene is regulated by any known regulatory molecule, preferably by Doxycycline.

46. The vector system according to claim 36, wherein the regulable expression cassette is inserted into the vector either in tandem or in opposite direction.

47. The vector system according to claim 36, wherein it comprises a first expression cassette comprising a CMV promoter and a second generation reverse tetracycline transactivator rtTA-M2 and a second expression cassette comprising a second generation tetracycline response promoter tight1 and nucleotide sequence coding for a sCAR-Fc fusion protein according to sequence 1 or a sequence inverse to sequence 1.

48. The vector system according to claim 36, wherein after transduction of an organism with said vector and after induction the transgene is expressed in a rate up to 500 ng in a ml blood plasma of an organism, preferably up to 700 ng/ml, preferably up to 1000 ng/ml, preferably up to 1500 ng/ml, preferably up to 2000 ng/ml, preferably up to 2500 ng/ml, preferably up to 2700 ng/ml, preferably up to 3000 ng/ml.

49. The vector system according to claim 36 for use as a medicament.

50. The vector system according to claim 36 for treatment of cells infected with a virus of the Picornavirus family, preferably for the treatment of meningitis, myocarditis, pancreatitis, hand, mouth and foot disease and Bornholm disease, or for treatment of CVB infected cells, preferably infected cardiac or pancreatic cells, or for treatment of cells infected with adenovirus, especially cells infected with adenovirus A, C-F.

51. The vector system according to claim 36 for treatment of cells infected with a virus of the Picornavirus family in combination with other viral inhibiting agents, preferably siRNA.

52. The vector system according to claim 50, wherein it is administered before, simultaneously or after infection of the virally infected cells, preferably in a dosage of 1×10¹⁰ to 1×10¹⁵ vector particles in case of in vivo treatment.

53. The vector system according to claim 36, wherein as a viral vector a vector selected from the group comprising an adenoviral vector, a replication deficient adenoviral vector, an adeno-associated virus (AAV), a retro virus vector, a reovirus vector, a herpes vector or a lentiviral vector having at least one deletion of at least one gene is used.

54. A composition comprising a vector system according to claim 36 and antiviral siRNAs.

55. The composition according to claim 54, wherein the siRNA comprises siRNA2 according to sequence 2 and siRNA4 according to sequence 3.

56. The composition according to claim 54 for use as a medicament.

57. The composition according to claims 54 for treatment of cells infected with a virus of the Picornavirus family, preferably for the treatment of meningitis, myocarditis, pancreatitis hand, mouth and foot disease and Bornholm disease, or for treatment of CVB infected cells, preferably infected cardiac or pancreatic cells, or for treatment of cells infected with adenovirus, especially cells infected with adenovirus A, C-F.

58. The composition according to claim 54, wherein it is administered to the cells before, simultaneously or after viral infection of the cells.

59. A method for treating infections caused by a virus of the Picornavirus family, preferably for treating meningitis, myocarditis, pancreatitis, hand, mouth and foot disease and Bornholm disease using a vector system according to claim 36.

60. The method according to claim 59 for treating CVB infected cells, preferably infected cardiac or pancreatic cells, or for treating cells infected with adenovirus, especially cells infected with adenovirus A, C-F.

61. A method for treating infections caused by a virus of the Picornavirus family, preferably for treating meningitis, myocarditis, pancreatitis, hand, mouth and foot disease and Bornholm disease using a composition according to claim 54.

62. The method according to claim 61 for treating CVB infected cells, preferably infected cardiac or pancreatic cells, or for treating cells infected with adenovirus, especially cells infected with adenovirus A, C-F.
